(19) United States
(12) Patent Application Publication
(10) Pub. No.: US 2025/0041204 A1
(43) Pub. Date: Feb. 6, 2025

(54) COMPOSITION COMPRISING LACTOBACILLUS CRISPATUS BACTERIAL LYSATES AND/OR BACTERIA FOR IMPROVING SKIN CONDITION

(71) Applicant: BIOTIX PTE LTD, Singapore (SG)

(72) Inventor: Davide PENNINO, Abingdon (GB)

(21) Appl. No.: 18/717,790

(22) PCT Filed: Dec. 9, 2022
(86) PCT No.: PCT/EP22/85164

§ 371 Date: Jun. 7, 2024

(30) Foreign Application Priority Data
Dec. 10, 2021 (SG) 10202113743T

(51) Int. Cl. A61K 8/99; A61K 35/747; A61P 17/00; A61P 17/04; A61P 17/06; A61Q 19/00; A61Q 19/08

(52) U.S. Cl. CPC A61K 8/99; A61K 35/747; A61P 17/00; A61P 17/04; A61P 17/06; A61Q 19/00; A61Q 19/08

(57) ABSTRACT

Disclosed herein are compositions for improving skin condition, wherein the composition comprises bacterial lysates and/or bacteria.

### FIELD OF THE INVENTION

The present invention relates to compositions comprising bacterial lysates and/or bacteria. The compositions are suitable for topical administration and may find use in improving the appearance of skin, improving skin health, preventing or treating skin inflammation, preventing skin aging and/or delaying or reversing the hallmarks of skin aging.

### BACKGROUND

The skin constitutes a barrier against external insults and the quality of this barrier is affected by external irritant agents (such as detergents, acids, bases, oxidants, reductants, concentration solvents, gases or toxic fumes), mechanical stresses (for example friction, impacts, abrasion, tearing of the surface, dust projection, particle projection, shaving or hair removal) or thermal or climatic imbalances (for example cold and dryness). These, and other, environmental factors can lead to skin irritation and inflammation which can be uncomfortable, disruptive to daily life, and painful.

Skin irritation is conventionally defined as a local, reversible, inflammatory reaction characterized by edema and erythema, which is induced after single or repeated contact of a chemical substance with the skin. Substances belonging to different classes of very different chemical products, such as keratinic solvents, dehydrating agents, or oxidant or reductant agents, may be considered to be irritants. Irritation of the skin is a very significant phenomenon, representing approximately 60% to 80% of clinical cases of non-allergic contact dermatitis.

Irritant contact dermatitis (ICD) can be defined as acute or chronic. Acute ICD is characterized principally by inflammation, whereas chronic ICD is characterized by hyperproliferation of keratinocytes and transitory hyperkeratosis. The biochemical events involved in skin irritation are complex and relatively little described. On contact between an irritant and the skin, the keratinocytes are the first cells to be damaged and activated by the chemical products. The keratinocytes play an essential part in initializing the inflammatory skin reaction through the release of numerous mediators and cytokines, which underlie a whole cascade of inflammation, ending in the clinical signs of ICD.

Atopic dermatitis, also known as eczema and atopic eczema, is one of the most common inflammatory disorders, affecting up to 20% of children and 10% of adults in high-income countries. The disorder is characterised by intense itching (pruritis) and recurrent eczematous lesions. Atopic dermatitis has a heterogeneous clinical presentation. The causes of atopic dermatitis are complex and multifactorial. There is a strong genetic component, with evidence for multiple mechanisms of genetic risk. Loss of-function mutations in the gene encoding filaggrin (FLG) are the most strongly and consistently reported genetic variants, supporting a vital role for the skin barrier, as filaggrin is a major structural protein in the epidermis. Although genetics are important in atopic dermatitis, the increasing global prevalence of the disorder highlights the role of environmental factors. Individuals with atopic dermatitis are at increased risk of having asthma, allergic rhinitis and food allergy, and could be at increased risk of a wide range of health and psychosocial outcomes. It has been broadly reported that the skin of Atopic Eczema may be affected by an imbalanced skin microbiome population (dysbiosis). For example, the skin of patients with Atopic Eczema may be enriched in the pro-inflammatory microbe Staphylococcus aureus, which promotes the onset of and aggravates the skin flares associated with the condition.

Psoriasis is a common skin disease affecting about 25 million people in North America and Europe and is probably the most prevalent immune-mediated skin disease in adults. It is an autoimmune disease affecting the skin that is triggered by an aberrantly activated cellular immune system and is similar to other immune-mediated disorders such as Crohn's disease, rheumatoid arthritis, multiple sclerosis, and juvenile-onset diabetes. The immunopathogenesis of Psoriasis involves both skin cells, keratinocytes, and immune cells, including dendritic cells, T cells, and in particular the T (h) helper 17 pathways. The main clinical symptoms displayed by patients suffering from psoriasis include erythema (rashes caused by injured and/or inflamed blood capillaries), induration, thickening of the skin, scaling, itchiness/pruritus and/or soreness.

Skin inflammatory conditions such as ICD, Psoriasis and Atopic Eczema are usually treated by topical corticosteroids as first-line treatment. However, these commonly used topical therapies can have significant side effects, including thickening of the skin, atrophy, striae, rosacea, perioral dermatitis, acne, purpura, hypertrichosis, pigment alteration, delayed wound healing, and exacerbation of skin infections. Individuals that are unresponsive to topical therapies may undergo systemic treatment with antibodies targeting TNF-alpha, Interleukin (IL-) 23, IL-17, and the IL-13 receptor. However, systemic treatments also show significant side effects, including opportunistic infections, itchiness and rashes.

The present invention has been devised in light of the above considerations, particularly the need for effective topical active ingredients with better safety profiles for use in preventing or treating skin disorders or reducing the severity of symptoms associated with skin disorders.

### SUMMARY OF THE INVENTION

The present inventors have found that healthy individuals have higher levels of commensal Lactobacillus crispatus on the skin compared to individuals that suffer from skin disorders. The inventors have also found that L. crispatus lysates have anti-inflammatory and skin-protective effects which, when applied to a subject, may prevent or treat skin inflammation. In addition, it has been found that L. crispatus lysates have anti-inflammatory effects on the mucous membrane of the vagina and penis, and in particular the glans of the penis. The inventors have further found that the levels of commensal L. crispatus decreases with age and as such compositions comprising L. crispatus, or an L. crispatus lysate, may be used to prevent or delay skin aging, or decrease the apparent age of skin. The invention thus provides a composition for topical administration, and cosmetic and clinical uses of said compositions, to prevent and treat skin inflammation, improve skin health, prevent or reduce pruritus, prevent or treat genital irritation, including vaginitis and balanitis, improve skin appearance, and prevent or delay skin aging, or decrease the apparent age of skin.

Accordingly, in a first aspect, the invention provides a composition for topical administration comprising;
i) a lysate of Lactobacillus crispatus or an active fraction thereof; and/or
ii) a population of Lactobacillus crispatus.

The lysate, active fraction and/or population may be present in an amount effective for improving the appearance of skin relative to skin prior to administration of the composition, improving skin health, preventing or treating skin inflammatory disorders, preventing or treating skin irritation, preventing or reducing pruritus, preventing or treating genital irritation, including vaginitis and balanitis, treating or preventing acne, delaying or preventing skin aging, or decreasing the apparent age of skin relative to skin prior to administration of the composition. Additionally or alternatively, the lysate, active fraction and/or population of L. crispatus may be present in an amount effective for treating irritative skin disease, preventing or reducing pruritus, preventing or treating genital irritation, including vaginitis and balanitis, dermatitis, and inflammatory skin disorders. For instance, the formulation comprises these active components in an amount that is effective for treating conditions such as irritant contact dermatitis, atopic dermatitis, eczema, atopic eczema, psoriasis, pruritus and/or skin irritation.

An effective amount of an L. crispatus population and/or L. crispatus lysate, or an active fraction thereof, may be determined using a functional assay. For example, an effective amount of the population, lysate or active fraction may be selected based on in vitro assays wherein the population, lysate or active fraction alters the expression of genes involved in one or more of collagen biosynthesis, collagen formation, extracellular matrix organisation, cholesterol regulation, cell junction organisation, collagen chain trimerization, Aryl-hydrocarbon signalling, and inflammation. In particular, the population, lysate or active fraction may be present in an amount sufficient to stimulate the Aryl-hydrocarbon-receptor pathway in vitro or in vivo, promote keratinocyte proliferation in vitro and/or protect keratinocytes from irritant induced cell death in an in vitro irritation model (e.g. as exemplified in Example 5).

The composition according to the first aspect may comprise one or more physiologically acceptable carriers, excipients, preservatives, fillers, anti-caking agents, anti-foaming agents, bulking agents, thickeners, gellants, emulsifying agents, surfactants, film-forming agents, propellants, pH adjusters, neutralising agents or stabilisers. One or more of these components may be added to aid in the formulation of a suitable topical composition for application to a subject, for example, cream, lotion, gel, mousse, solid stick, spray, ointment, or milk.

A second aspect of the invention provides a cosmetic method of improving the appearance of skin, improving skin health, preventing or reducing skin irritation, delaying or preventing skin aging, reducing or preventing acne, or decreasing the apparent age of skin relative to skin before administration of the composition. The cosmetic method comprises the step of applying the composition according to the first aspect to the skin of a subject. In some embodiments, the skin may appear more plump, smooth, firm, and/or hydrated following administration of the composition. In some embodiments the skin may appear less red, flaky, rough, and/or wrinkled following administration of the composition. In some embodiments, the method comprises applying the composition every 1 to 7 days. In some embodiments, the method comprises applying the composition once a day. In some embodiments, the composition may be applied 1 or more times a day, for example twice a day. In some embodiments, the appearance of skin will be improved, skin irritation reduced, and/or skin aging delayed, prevented or reversed after 1 day to 14 weeks. In some embodiments, the appearance of skin will be improved, skin irritation reduced, and/or skin aging delayed, prevented or reversed after 1 day, 2 days, 3, days, 4, days, 5, days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks.

A third aspect of the invention provides a composition according to the first aspect of the invention for use in a therapeutic method of improving the appearance of skin relative to skin prior to administration of the composition, improving skin health, preventing or treating skin irritation, preventing or reducing pruritus, preventing or treating genital irritation, including vaginitis and balanitis, treating or preventing acne, delaying or preventing skin aging, or decreasing the apparent age of the skin relative to skin before administration of the composition. Additionally or alternatively, the composition may be used to treat an inflammatory skin condition, an irritative skin disease and/or dermatitis, e.g. irritant contact dermatitis, atopic dermatitis, eczema, atopic eczema, psoriasis, pruritus and/or skin irritation. The method comprises applying the composition to the skin of a subject in need thereof. In some embodiments, the skin irritation may be caused by inflammatory skin irritation, irritant contact dermatitis, atopic dermatisis, eczema, atopic eczema, and/or psoriasis. In some embodiments, the method comprises applying the composition every 1 to 7 days. In some embodiments, the method comprises applying the composition once a day. In some embodiments, the composition may be applied 1 or more times a day, for example twice a day. In some embodiments, the appearance of skin will be improved, skin irritation reduced, pruritus reduced, genital irritation reduced, symptoms of balanitis or vaginitis reduced, and/or skin aging delayed, prevented or reversed after 1 to 14 weeks. In some embodiments, the appearance of skin will be improved, skin irritation reduced, pruritus reduced, genital irritation reduced, symptoms of balanitis or vaginitis reduced, and/or skin aging delayed, prevented or reversed after 1 day, 2 days, 3, days, 4, days, 5, days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks.

A fourth aspect of the invention provides a method of treating inflammatory skin disorders, irritative skin diseases, pruritus, genital irritation, balanitis, vaginitis, and/or dermatitis, e.g. irritant contact dermatitis, atopic dermatitis, eczema, atopic eczema, psoriasis, and/or skin irritation caused by inflammatory skin irritants, improving skin health, preventing or treating skin irritation, treating or preventing acne, delaying skin aging and/or reversing skin aging in a subject. The method comprises administering the composition according to the first aspect to the subject.

In some embodiments of the cosmetic method of the second aspect, the composition for use according to the third aspect, or the method for treating a subject according to the fourth aspect, the composition may alter the expression of genes involved in one or more of collagen biosynthesis, collagen formation, extracellular matrix organisation, collagen chain trimerization, Aryl-hydrocarbon signalling, anti-apoptotic molecules, cell proliferation and inflammation.

In some embodiments of the cosmetic method of the second aspect, the composition for use according to the third aspect, or the method for treating a subject according to the fourth aspect, the composition increases the expression of genes involved in one or more of collagen biosynthesis, collagen formation, extracellular matrix organisation, collagen chain trimerization, Aryl-hydrocarbon signalling, anti-inflammatory or anti-apoptotic molecules, and/or decreases expression of pro-inflammatory molecules.

In some embodiments of the cosmetic method of the second aspect, the composition for use according to the third aspect, or the method for treating a subject according to the fourth aspect, the composition activates the Aryl-hydrocarbon-receptor pathway.

In a fifth aspect is provided a method for altering the expression of genes involved in one or more of collagen biosynthesis, collagen formation, extracellular matrix organisation, collagen chain trimerization, Aryl-hydrocarbon signalling, anti-apoptotic molecules, cell proliferation and inflammation in a subject. In some embodiments, the method activates the Aryl-hydrocarbon-receptor pathway in the subject. In some embodiments, the subject is human. In some embodiments, the method is a therapeutic method. In some embodiments, the method is a cosmetic method. In some embodiments, the method treats inflammatory skin disorders, irritative skin diseases, pruritus, genital irritation, balanitis, vaginitis, and/or dermatitis, e.g. irritant contact dermatitis, atopic dermatitis, eczema, atopic eczema, psoriasis, and/or skin irritation caused by inflammatory skin irritants, improves skin health, prevents or treats skin irritation, treats or prevents acne, delays skin aging and/or reverses skin aging in the subject.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1A shows the skin of patients suffering from atopic eczema and psoriasis have reduced levels of L. crispatus compared to the skin of healthy individuals.

FIG. 1B shows the population of L. crispatus present on the skin of healthy individuals decreases with age.

FIG. 2 shows L. crispatus regulates the expression of genes associated with collagen biosynthesis, collagen formation, AhR signalling and inflammation.

FIG. 3 shows L. crispatus lysates positively regulate the Ary-hydrocarbon receptor (AhR) pathway and cause increased expression of the CYP1A1 and AhRR genes which are under the direct control of AhR.

FIG. 4A shows the results of an in vitro irritation model demonstrating that L. crispatus lysate protects keratinocytes from irritant-induced cell death.

FIG. 4B shows that L. crispatus lysates provide greater protection to keratinocytes from irritant induced cell death compared to other commonly used skin care active agents.

FIG. 5 shows L. crispatus lysates protect healthy volunteers from SDS-mediated skin irritation and that L. crispatus lysates do not cause skin irritation.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

First-line corticosteroid treatments for inflammatory skin conditions such as ICD, psoriasis and atopic eczema can have significant side-effects including thickening of the skin atrophy, striae, rosacea, perioral dermatitis, acne, purpura, hypertrichosis, pigment alteration, delayed wound healing, and exacerbation of skin infections. Similarly, systemic treatment with antibodies can also show significant side effects including infection, itchiness (pruritus) and rashes. Consequently, there is a need for the development of safe and effective compositions for alleviating skin irritation.

The invention relates to the finding that Lactobacillus crispatus (L. crispatus) colonises the skin of healthy individuals and is decreased in individuals with skin inflammation disorders, such as atopic eczema and psoriasis, and is decreased in older individuals, indicating L. crispatus may play a role in maintaining normal skin health and loss of commensal L. crispatus may contribute to skin aging. The invention, therefore, provides a composition for topical administration comprising a population of L. crispatus and/or a lysate of L. crispatus to improve the appearance of skin; improve skin health; prevent and/or treat skin inflammatory disorders; prevent or reduce pruritus, prevent or reduce genital irritation, prevent or reduce symptoms of balanitis or vaginitis, or prevent, delay and/or reverse skin aging.

L. crispatus is a common species of bacteria known to colonize the vagina and vertebrate gastrointestinal tract. To date, L. crispatus has been recognised as a useful probiotic to treat recurrent urinary tract infections in women. However, the use of an L. crispatus lysate, or the use of L. crispatus as a probiotic, to improve the appearance of skin, treat skin inflammatory disorders, prevent or reduce pruritus and/or prevent/reverse skin aging has not previously been reported. L. crispatus is publicly available, e.g. from microbial deposits such as ATCC, NCIMB, DSMZ.

In some embodiments, the lysate of L. crispatus may be the total lysate, including the intracellular components of the cells and the cell walls and membranes of the cells. Cell lysis can be carried out by any suitable method such as, for example, sonication, osmotic shock, thermal shock, by ultrasound, mechanical disruption, for example French press, liquid homogenization, or centrifugal mechanical strain. In some embodiments, the lysate may be an active fraction of the lysate. An active fraction may comprise one or more isolated components of the lysate which still improves the appearance of skin, improves skin health, prevents and/or treats skin inflammation, prevent or reduce skin pruritus, prevent or reduce genital irritation, prevent or reduce symptoms of balanitis or vaginitis, prevents or delays skin aging or decreases the apparent age of the skin when applied to a subject as part of a topical composition. For example, an active fraction may contain one or more metabolites, proteins, carbohydrates, nucleic acids or lipids derived from the lysate. An active fraction of the lysate may also be a purified fraction lacking one or more components of the total lysate and still improves the appearance of skin, improves skin health, prevents and/or treats skin inflammation, prevent or reduce skin pruritus, prevent or reduce genital irritation, prevent or reduce symptoms of balanitis or vaginitis, prevents or delays skin aging or decreases the apparent age of the skin when applied to a subject as part of a topical composition. For example, an active fraction may have had one or more metabolites, proteins, carbohydrates, lipids, or nucleic acids (e.g. RNA or DNA) removed from the total lysate. Removal of DNA and/or RNA from a total lysate may be carried out by any suitable technique known in the art, for example, through precipitation, centrifugation, DNAse treatment and/or RNAse treatment.

In some embodiments, the composition may comprise an L. crispatus lysate, or active fraction thereof, from 0.0001% to 100% by weight, from 0.001% to 75% by weight, 0.01% to 50% by weight, or from 0.1% to 25% by weight in relation to the total weight of the composition. In some embodiments, the composition may comprise an L. crispatus lysate, or an active fraction thereof, from 0.0001% to 100% by volume, from 0.001% to 75% by volume, 0.01% to 50% by volume, or from 0.1% to 25% by volume in relation to the total volume of the composition. In some embodiments, the composition may comprise an L. crispatus lysate, or an active fraction thereof, of up to 1%, 2%, 5%, 10%, 15%, 20%, 30%, 40%, 50%, 60%, 70%, 90% or 100% by weight or by volume in relation to the total weight of the composition. In some embodiments, the composition may comprise an L. crispatum lysate, or an active fraction thereof, of at least 1%, 2%, 5%, 10%, 15%, 20%, 30%, 40%, 50%, 60%, 70%, 80%, 90% or 100% by weight or by volume.

In some embodiments, the concentration of the lysate per gram of composition corresponds to the amount of lysate produced from a population of L. crispatus equal to 10² to 10¹¹, 10⁵ to 10¹⁰, or 10⁷ to 10⁹ cfu/g.

In some embodiments, the composition may comprise L. crispatus at a concentration from 10² to 10¹¹ cfu/g, 10⁵ to 10¹⁰ cfu/g, or 10⁷ to 10⁹ cfu/g.

In some embodiments, the composition may comprise a population of L. crispatus from 0.0001% to 100% by weight, from 0.001% to 75% by weight, 0.01% to 50% by weight, or from 0.1% to 25% by weight L. crispatus in relation to the total weight of the composition. In some embodiments, the composition may comprise a population of L. crispatus from 0.0001% to 100% by volume, from 0.001% to 75% by volume, 0.01% to 50% by volume, or from 0.1% to 25% by volume in relation to the total volume of the composition. In some embodiments, the composition may comprise an L. crispatus population of up to 1%, 2%, 5%, 10%, 15%, 20%, 30%, 40%, 50%, 60%, 70%, 90% or 100% by weight or by volume in relation to the total weight of the composition. In some embodiments, the composition may comprise and L. crispatum population of at least 1%, 2%, 5%, 10%, 15%, 20%, 30%, 40%, 50%, 60%, 70%, 80%, 90% or 100% by weight or by volume.

In some embodiments, the composition may comprise a lysate, or active fraction thereof, derived from an isolated strain of L. crispatus, for example the L. crispatus strain VPI3199. In some embodiments, the lysate, or active fraction thereof, may be derived from an isolated strain of L. crispatus, for example the L. crispatus strain VPI3199. In some embodiments, the composition may comprise an isolated strain of L. crispatus, for example the L. Crispatus strain VPI3199. In some embodiments, the population of L. crispatus is L. crispatus VPI3199. The L. crispatus VPI3199 strain has been described in Lactobacillus crispatus (Brygoo and Aladame 1953) Moore and Holdeman (1970).

A composition of the current invention comprises an effective amount of an L. crispatus lysate, or a population of L. crispatus, for improving the appearance of the skin, improving skin health, preventing or treating skin irritation, preventing or reducing skin pruritus, preventing or reducing genital irritation, preventing or reducing symptoms of balanitis or vaginitis, and/or preventing, delaying or reversing skin aging. A composition of the current invention can take any suitable form for topical administration and may include pharmaceutically or cosmetically suitable carriers, excipients, preservatives, fillers or stabilisers which are well known in the art. For example, the composition may comprise one or more excipients selected from isopropanol, hydroxypropylcellulose, glyceryl stearate, cetylstearyl alcohol, glycerol, cetyl alcohol liquid petrolatum, isopropyl myristate, Methyl POB, Propyl POB

Suitable composition formulations for topical administration are well known in the art and include creams, lotions, gels, mousses, solid sticks, sprays, ointments, or milks. Suitable techniques for preparing such formulations are well known in the art and can be found, for example, in Williams, D. and Schmitt, W., 1996. Chemistry and Technology of the Cosmetics and Toiletries Industry. Dordrecht: Springer Netherlands, Harry, R. and Rieger, M., 2000. Harry's cosmeticology. New York: Chemical Pub. Co., and Remington, J. and Gennaro, A., 2000. Remington the science and practice of pharmacy. Baltimore, Md.: Lippincott Williams & Wilkins.

The composition of the invention may also comprise other topically useful components which may enhance or complement the activity of the L. crispatus population, lysate or active fraction. The choice of additional topically useful components will depend on the intended formulation and use of the composition. Topically useful components which are standard in the art can be found, for example, in

Gottschalck, T. and McEwen, G., 2004. International cosmetic ingredient dictionary and handbook. Washington, D.C.: Cosmetic, Toiletry, and Fragrance Association.

Examples of topically useful components that may be used with the compositions of the invention include, but are not limited to: fragrances or essential oils; pigments or colorants; formulation aids such as anti-caking agents, anti-foaming agents, fillers and bulking agents, thickeners, gellants, structuring agents and emulsion stabilisers; surfactants and emulsifiers; film-forming agents to enhance adhesion and retention on the intended target (e.g. the skin); propellants, preservatives and pH adjusters and neutralising agents.

Other topically useful components may provide a further benefit (i.e. skin benefit agents, or active agents) to the keratinous surface to which the composition will be applied. These include, but are not limited to astringents, such as clove oil, menthol, camphor, eucalyptus oil, eugenol, menthyl lactate, witch hazel distillate; antioxidants or free-radical scavengers, such as ascorbic acid, its fatty esters and phosphates, tocopherol and its derivatives, N-acetyl cysteine, xacid and lipoic acid; anti-acne agents, such as salicylic acid and benzoyl peroxide; antimicrobial or antifungal agents such as caprylyl glycol, triclosan, phenoxyethanol, erythromycin, tolnaftate, nystatin or clortrimazole; chelating agents, such as EDTA; topical analgesics, such as benzocaine, lidocaine or procaine; anti-aging/anti-wrinkle agents, such as retinoids or hydroxy acids; skin lightening agents, such as licorice, ascorbyl phosphates, hydroquinone or kojic acid; skin-conditioning agents, such as humectants, including miscellaneous and occlusive; anti-irritants, such as cola, bisabolol, aloe vera or panthenol; anti-inflammatoirech as hydrocortisone, clobetasol, dexamethasone, prednisone, acetyl salicylic acid, glycyrrhizic acid or glycyrrhetic acid; anti-cellulite agents, such as caffeine and other xanthines; humectants, such as alkylene polyols or hyaluronic acid; emollients, such as oily esters or petrolatum; sun protecting agents (organic or inorganic), such as avobenzone, oxybenzone, octylmethoxycinnamate, titanium dioxide or zinc oxide; exfoliating agents (chemical or physical), such as N-acetyl glucosamine, mannose phosphate, hydroxy acids, lactobionic acid, peach kernels, or sea salts; self-tanning agents, such as dihydroxyacetone; and biologically active peptides, such as palmitoyl pentapeptide or argireline. These supplemental skin benefit agents will be used in the amounts normally known to be effective for that active when used for the intended purpose.

Healthy skin may be defined as compared to diseased skin. Healthy skin is characterized by a fully formed and competent epidermal barrier. Hallmarks of healthy skin include epithelial integrity, moisturized, high elasticity, functional wound healing, non-itchy, non-inflamed and a lack of redness or erythema.

Skin health and appearance can be assessed based on a number physiological parameters, including, hydration or dryness, transepidermal water loss (TEWL), erythema index, elasticity, laxity, skin friction, redness and scaling. Skin health and appearance can also be assessed based on the presence or absence of symptomatology of skin disorders (e.g. eczema, acne, rosacea) and the presence or absence of dark spots.

An improvement in skin health and appearance may correlate with an increase in plumpness and/or smoothness and may appear less wrinkled. An improvement in skin health and appearance may also correlate with a decrease in the flakiness and/or roughness of the skin. An improvement in skin health and appearance may also correlate with a decrease in the intensity of redness or rash intensity. An improvement in skin health and appearance may also correlate with a decrease in the number of wrinkles in the skin.

For example, a decrease in extracellular collagen in skin health can be associated with a decrease in skin hydration and aging skin. Loss of water from the outer most layer of the skin, the stratum corneum, can also lead to dysregulation of normal desquamation (normal shedding of the outer-most dead skin cells) leading to the visible appearance of dry and/or flaky skin further impacting the physical appearance and health of the skin.

Compositions of the present invention may improve the appearance of skin, improve skin health, decrease or prevent inflammatory skin diseases, skin irritation, prevent or reduce skin pruritus, prevent or reduce genital irritation, prevent or reduce symptoms of balanitis or vaginitis, prevent skin aging or reverse skin aging by altering gene expression, for example by altering gene expressing in cells of the epidermis, dermis, and/or immune system. For example, the composition may cause an increase or a decrease in gene expression upon topical administration to the skin. In particular, the composition may alter the expression of genes associated with collagen biosynthesis, collagen formation, retinol biosynthesis, extracellular matrix organisation, collagen chain trimerization, Aryl-hydrocarbon signalling, and inflammation.

In some embodiments, the composition of the invention may contain a lysate, active fraction, or population in an amount sufficient to alter the expression of genes involved in one or more of collagen biosynthesis, (such as retinoid acid receptor (RAC), cytochrome (CYPA1A1), and collagen genes such as COL1, COL2), collagen formation, extracellular matrix organisation, collagen chain trimerization, Aryl-hydrocarbon signalling (such as AhR repressor), anti-apoptotic molecules, cell proliferation and inflammation when applied to skin cells, keratinocytes, fibroblast or skin immune cells in vitro or in vivo. In particular, the lysate, active fraction or population may be present in an amount sufficient to activate the Aryl-hydrocarbon-receptor pathway when applied to skin cells, keratinocytes, fibroblast and skin immune cells in vitro and in vivo. Gene expression may be measured using any standard technique known in the art, for example, real-time PCR, Northern blotting, Western blotting or DNA microarrays may used to determine gene expression.

Collagen is of particular importance in maintaining skin hydration, and thus maintaining good skin health and appearance. Collagen is the most abundant component of the extracellular matrix and helps maintain skin structure to support a smooth, firm, and strong skin. A decrease in collagen abundance correlates with a decrease in skin hydration, elasticity and density, and an increase in skin roughness and laxity, leading to the appearance of skin aging and/or a decrease in skin appearance and health. The composition may prevent or reverse skin aging, improve the appearance of skin, and/or improve skin health by promoting collagen biosynthesis and collagen formation. For example, the composition may promote expression of genes associated with collagen biosynthesis and formation and/or inhibit expression of genes associated with a decrease in collagen biosynthesis and formation. In particular, the composition may promote expression of genes involved in retinol biosynthesis. Retinol promotes an anti-aging effect by activating the retinoid acid receptor (RAC) in the epidermis which induces expression of genes such as CYPA1A1 (cytochrome) and collagen genes, such as COL1 and COL3. As result the composition may reduce the appearance or number of wrinkles and improve elasticity of the skin.

The abundance of collagen in the extracellular matrix of the skin can decrease with age. The decrease in collagen abundance in the skin can be accompanied by changes in the physical appearance of the skin and may include the skin appearing more wrinkled, less elastic, laxer, less hydrated, and rough textured.

As discussed above, the composition may reverse the appearance of skin aging by promoting expression of collagen and the abundance of collagen present in the extracellular matrix of the skin. As a result of the increased collagen in the extracellular matrix of the skin the abundance and/or appearance of wrinkles may be reduced; the skin may appear less lax and/or have increased elasticity; and/or the skin may appear more smooth, plump, and/or hydrated.

The composition may therefore be considered an “anti-aging” composition in that the composition may prevent or delay the increase in apparent age of the skin. A delay in skin aging may be characterised as a delay in the appearance of wrinkles in the skin that would otherwise appear had the composition of the invention not been applied. A delay in skin aging may also be associated with a delay in the rate at which the skin loses elasticity, increases in laxity, loses hydration and/or appears rougher as a consequence of aging. Similarly, preventing skin aging can be characterised by the prevention of the appearance of wrinkles, preventing the skin losing elasticity, preventing the skin becoming more lax, preventing loss of skin hydration, and/or preventing the skin becoming rough as a consequence of aging.

A delay in skin aging, or prevention of skin aging, may be determined by any well-known method in the art. For example, the physical characteristics of the skin (e.g. wrinkling, loss of elasticity, increased laxity, decrease in hydration, skin roughness etc.) in a sample group of individuals can be measured before applying the composition one or more times and then repeating the measurements following administration of the composition. The physical characteristics of the skin of a control group of individuals can also be measured over the same period of time as the sample group and the rate of change in the progression of skin aging can be compared between the two groups. A decreased rate of skin aging in the sample group compared to the control group would indicate the composition delayed or prevented an increase in the apparent age of the skin in the sample group. A delay or prevention of skin aging may also be assessed by applying the composition to a defined test area of an individual over a period of time. The rate of skin aging of the test area can then be compared to the rate of skin aging in a control area of the same individual that was not exposed to the composition. A decreased rate of skin aging in the test area compared to the control area would indicate the composition delayed or prevented an increase in the apparent age of the skin in the test area.

The composition may also reduce or reverse the apparent age of the skin. “Reversing” skin aging refers to a reduction in the presentation of characteristics associated with aging skin (e.g. wrinkling, loss of elasticity, increased laxity, decrease in hydration, skin roughness etc.). Thus, reversing or reducing skin aging means to reduce the apparent age of the skin. For example, a reduction in the apparent age of skin may be characterised by a reduction in the number of wrinkles present on the skin after applying the composition of the invention compared to the skin before the composition was applied. A reduction in the apparent age of the skin may also be associated with the skin becoming more elastic, less lax, more hydrated, and/or the skin appearing smoother and/or less rough. A reduction or reversal of the apparent age of the skin may be measured by comparing the physical characteristics of an area of skin (e.g. number and/or severity of wrinkles, skin elasticity, laxity, roughness, hydration etc.) before application of the composition to that of the same area of skin after applying the composition to the skin one or more times. A decrease in, for example, the number of wrinkles, increased elasticity, decreased laxity, decreased roughness and/or increased hydration will indicate the composition has reversed or reduced the apparent skin age,

Erythema is a rash caused by injured or inflamed blood capillaries. Erythema is characterised by a reddening of the skin in the affected area and may be considered as a sign of a decrease in skin health and/or a decrease in the appearance of the skin. Erythema may arise as a symptom of a skin inflammatory disorder, such as eczema, psoriasis, and irritant contact dermatitis. The composition according to the first aspect of the invention may improve skin health and/or improve the appearance of the skin by reducing the intensity of the redness associated with erythema. The composition may reduce the redness associated with erythema by reducing the inflammatory response at the affected site. For example, the composition may reduce inflammation by modulating the expression of genes associated with the AP-1 and AP-2 inflammation pathways and/or Aryl-hydrocarbon receptor pathways.

In addition to playing a role in modulating the inflammation, the Aryl-hydrocarbon receptor pathway is known to play a role in maintaining the barrier function of the skin and limiting transepidermal water loss thereby maintain skin hydration (Haas et al., 2016). The composition according to the first aspect of the invention may therefore improve skin appearance, improve skin health, reverse the appearance of skin aging, or prevent the appearance of skin aging by modulating gene expression of genes associated with the Aryl-hydrocarbon receptor signalling pathway thereby reducing the levels of transepidermal water loss.

Methods for measuring the physiological parameters and characteristics of the skin, such as hydration, transepidermal water loss, erythema index, elasticity, erythema, darkness, roughness, extensibility, resistance, radiance, dullness, dryness, pH, pigmentation are well known in the art and can be found, for example, in Agache, A., 2016. Agache's measuring the skin. Cham: Springer. For example, skin firmness, elasticity and smoothness can be measured using techniques standard in the art, for example, using cutometry. Skin hydration may be determined using a capacitance meter which determines the water content of the skin and/or by measuring the rate of transepidermal water loss. The degree of redness, flakiness, roughness, and degree of wrinkling may be assessed by visual inspection of the skin, for example by a trained dermatologist.

The lysate, active fraction, or population may be present in the composition in amount sufficient to promote keratinocyte proliferation in vitro and in vivo. Standard cell proliferation assays known in the art may be used to determine if the lysate, active fraction, or population stimulate proliferation of cells in vivo. For example, colorimetric technics, flow cytometry or imaging can be used to assay the rate of cell proliferation in a population of cells exposed to the composition compared to cells that have not been exposed to the composition.

The lysate, active fraction, or population may be present in the composition in amount sufficient to protect keratinocytes from irritant induced cell death in vitro and in vivo. An in vitro irritation assay, for example the assay described in Example 5, may be used to determine if the lysate, active fraction, or population is present in an amount sufficient to protect keratinocytes from irritant induced cells death. A reduction of cell death may be determined by comparing the rate of cell death in population of cells exposed to an irritant (e.g. SDS) and the composition of the invention to cells exposed to the irritant alone. The lysate, active fraction, or population may be present in the composition in amount sufficient to prevent or reduce skin inflammation, for example irritant-induced skin inflammation. This may be assessed using the model for irritant-induced inflammation described in Example 6. A reduction of irritant-induced inflammation of the skin may be determined relative to the amount of inflammation seen in a subject exposed to the irritant without exposure to the composition of the invention.

In a second aspect, the present invention relates to a cosmetic method comprising applying the composition according to the first aspect to the skin of a subject. The method may improve the appearance of the skin of a subject relative to the appearance of the skin before application of the composition. In addition to, or instead of, the method may decrease the apparent age of the skin of a subject relative to the apparent age of the skin before application of the composition. The composition may also be used to prevent, or reduce the onset of, an increase in the apparent age of skin relative to the increase in the apparent age of skin had the composition not been applied to the skin.

An improvement in the appearance of skin, or a decrease in the apparent age of skin, can be associated with an increase in the apparent plumpness of the skin and/or an increase in the smoothness of the skin (i.e. the skin may appear less wrinkled) compared to the appearance of the skin before administration of the composition. An improvement in the appearance of the skin can also be associated with a decrease in the redness, flakiness or roughness of the skin compared to the appearance of the skin before administration. The skin may also appear more hydrated following administration of the composition compared to the appearance before administration. Methods for measuring the plumpness, smoothness, redness, flakiness, roughness, hydration, and wrinkle severity of skin are well known in the art.

Administration is preferably in an effective amount, this being sufficient to show benefit to the individual, i.e. an improvement in the appearance of the skin or an apparent decrease in the age of the skin. The composition may be applied to the skin at the discretion of the subject. For example, the composition may be applied between and 1 and 10 times a day. An increase in the appearance of the skin, or a decrease in the apparent age of the skin may be apparent following continued use of the composition over a period of time. For example, an improvement in the appearance of the skin, or a decrease in the apparent age of the skin, may be apparent following continued use of the composition for between 1 day and 1 year.

In a third aspect, the present invention relates to the composition according to the first aspect for use in a method of treating inflammatory skin diseases, irritative skin diseases, pruritus, genital irritation, balanitis, vaginitis, acne, and/or dermatitis, e.g. irritant contact dermatitis, atopic dermatitis, eczema, atopic eczema, psoriasis, and/or skin irritation is caused by a skin irritant. In a fourth aspect, the present invention relates to a method of treating an inflammatory skin disease, irritant contact dermatitis, atopic dermatitis, eczema, atopic eczema, psoriasis, pruritus, genital irritation, balanitis, vaginitis, acne and/or skin irritation caused by inflammatory skin irritants in a subject in need thereof, the method comprising administering the composition according to the first aspect to the subject. In some embodiments of the third and fourth aspects, the composition is for use in a method of treating or preventing acne. In some embodiments, the composition comprises a lysate of Lactobacillus crispatus or an active fraction thereof for use in a method of treating or preventing acne. In some embodiments of the third and fourth aspects, wherein the composition comprises a population of Lactobacillus crispatus, the composition is not for use in a method of treating or preventing acne.

In the third and fourth aspects, treating may include preventing, alleviating or reducing symptoms associated with a disease or disorder; and/or curing or preventing onset of a disease or disorder. Atopic eczema can affect any part of the skin but is most commonly associated with the creases of joints at the elbows and knees as well as the wrists and neck. Coin-sized areas of inflammation on the limbs as well as small bumps that coincide with hair follicles are also common in subjects with atopic eczema. The affected skin is usually red, dry and itchy. Affected skin may also weep, blister, become crusty, scale or thicken.

Due to the associated itchiness, it is common for a subject suffering from atopic eczema to scratch the affected area leading to further damage to the skin which may cause bleeding. In areas that are repeatedly scratched, the skin may thicken (lichenification) leading to more intense itching and further damage. When atopic eczema is in a very active phase, the affected area may become moist and weep and may be associated with the development of small water blisters, especially on the hands and feet. Topical administration of the composition according to the invention may reduce one or more of the symptoms associated with atopic eczema. For example, the composition may reduce inflammation, redness, itchiness (pruritus) or dryness of affected areas. The composition may also prevent skin from becoming inflamed, red, itchy, or dry due to atopic eczema. Reducing or preventing symptoms associated with atopic eczema may prevent a subject from scratching and further damaging the skin (e.g. prevent lichenification and/or breaking the skin barrier). Psoriasis is a common chronic inflammatory skin disorders characterized by heterogenous clinical manifestation. Clinical manifestation include well-demarcated, symmetrical erythematous plaques with adherent silvery scale. The psoriasis area and severity index (PASI) is a quantitative rating score for measuring the severity of psoriatic lesions based on area coverage and plaque appearance and may be used to assess severity of symptoms pre- and post-treatment. The PASI score takes into consideration both the plaque characteristics and percentage area of affected body parts covered by the psoriatic lesions. Plaque characteristics may be judged on levels of erythema, induration/thickening of the skin, and scaling. Topical administration of the composition according to the first aspect of the invention may reduce the severity of erythema, induration/thickening of the skin, scaling, itchiness and/or soreness caused by psoriatic lesions. Topical administration of the composition may reduce the total area cover by psoriatic lesions. Therefore, topical administration of the composition may reduce the PASI score of a patient.

Contact dermatitis is a common inflammatory skin condition characterized by erythematous and pruritic skin lesions that occur after contact with a foreign substance. There are two forms of contact dermatitis: irritant and allergic. Irritant contact dermatitis is caused by the non-immune-modulated irritation of the skin by a substance, leading to skin changes. Allergic contact dermatitis is a delayed hypersensitivity reaction in which a foreign substance comes into contact with the skin; skin changes occur after reexposure to the substance. The most common substances that cause contact dermatitis include poison ivy, nickel, and fragrances. Contact dermatitis usually leads to erythema and scaling with visible borders. Itching and discomfort may also occur. Acute cases may involve a dramatic flare with erythema, vesicles, and bullae; chronic cases may involve lichen with cracks and fissures. Topical administration of the composition may reduce the severity of the symptoms associated with contact dermatitis, including erythema, scaling, and itching. Topical administration of the composition may prevent a patient from developing symptoms associated with contact dermatitis, for example, preventing the onset of itching, erythema or scaling.

Irritant contact dermatitis is characterized by inflammation of the skin due to direct contact with a substance on the surface of the skin. It is usually caused by substances such as solvents or other chemicals that can irritate the skin. Exposure can cause red spots and itching on the affected skin area.

Irritant contact dermatitis occurs in 80% of all sufferers of contact dermatitis while allergic contact dermatitis only occurs about 10-20%. This condition involve a combination of endogenous and exogenous factors, which triggers a pathophysiological cascade of skin barrier disruption, cellular damage to the keratinocyte membrane and pro-inflammatory mediator release, resulting in a clinical presentation may be characterized by erythema, oedema, local necrosis starting soon after the exposure to the irritant and can develop in pustular and acneiform dermatitis. In contrast to Allergic Contact Dermatitis, ICD patients do not develop a skin allergy and result negative to patch testing. Topical administration of the composition may reduce the severity of the symptoms associated with irritant contact dermatitis, including itching and erythema. Topical administration of the composition may prevent a patient from developing symptoms associated with irritant contact dermatitis, for example, preventing the onset of itching, or erythema.

Genital irritation is a common condition affecting both women and men with a variety of underlying causes, such as a bacterial or yeast infection or as a result of an allergic reaction. Vaginitis is a condition whereby the vagina becomes infected and/or inflamed. Vaginitis is characterised by soreness and swelling in and around the vagina which can lead to discomfort and itching (pruritis). Vaginitis may also include inflammation of the vulva (termed vulvovaginitis). The term “vaginosis” used herein is used to also encompass vulvovaginitis. Symptoms of vaginosis include, for example, irritation, pruritus, and erythema. The underlying cause of vaginitis may be a bacterial (e.g. chlamydia or gonorrhoea), yeast (e.g. candida), viral (e.g. Herpes simplex virus or human papillomavirus) or parasitic (e.g. trichomoniasis) infection. Vaginitis may also develop as the result of contact with an allergen resulting in an allergic reaction in and around the vagina. Vaginitis may also be caused by exposure of the vagina to an irritant (i.e. a non-allergic reaction).

Balanitis is a condition whereby the glans (head) of the penis becomes inflamed. Patients suffering from balanitis normally present with a red, swollen, itchy and sore glans. Symptoms of balanitis include, for example, pain, tenderness, and pruritus which may be associated with erythematous lesions on the glans and/or the foreskin of the penis. The underlying cause of balanitis may be a bacterial (e.g. chlamydia or gonorrhoea), yeast (e.g. candida), viral (e.g. Herpes simplex virus or human papillomavirus) or parasitic (e.g. trichomoniasis) infection. Balanitis may also develop as the result of contact with an allergen resulting in an allergic reaction in and around the glans. Balanitis may also be caused by exposure of the penis to an irritant (i.e. a non-allergic reaction) or through poor hygiene.

Topical administration of the composition of the invention to genitals may reduce the severity of the symptoms associated with genital irritation or prevent the onset of the symptoms associated with genital irritation. For example, topical administration of the composition to the vagina may reduce the severity of symptoms, or prevent the onset of symptoms, associated with vaginitis. For example, the severity of itching and/or erythema may be reduced following topical administration of the composition to the vagina of a patient suffering from vaginitis. Equally, topical administration of the composition to the penis may reduce the severity of symptoms, or prevent the onset of symptoms, associated with balanitis. For example, the severity of itching and/or erythema may be reduced following topical administration of the composition to the penis of a patient suffering from balanitis.

The method comprises topical administration of the composition in a therapeutically effective amount to the skin of a subject. Administration is preferably in a “therapeutically effective amount”, this being sufficient to show benefit to the individual. The actual amount administered, and rate and time-course of administration, will depend on the nature and severity of the disease being treated. For example, the composition may be applied between 1 and 10 times per day. The composition may also be applied as needed by a subject, for example, on presentation of a symptom (e.g. pruritis) associated with inflammatory skin irritation, irritant contact dermatitis, atopic dermatitis, eczema, atopic eczema, genital irritation, vaginitis, balanitis, or psoriasis.

Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

It will be understood by the skilled person that compositions for use in accordance with the third aspect of the invention will comprise the active ingredients and, if any, pharmaceutically acceptable excipients and carriers of a suitable pharmaceutical grade.

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as skin irritation and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

The term “carrier” refers to diluents, binders, lubricants and disintegrants. Those with skill in the art are familiar with such pharmaceutical carriers and methods of compounding pharmaceutical compositions using such carriers.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients used in the composition, i.e. the lysate, fraction and/or population of Lactobacillus crispatus. Thus, the skilled person will appreciate that compositions are provided wherein there is no incompatibility between any of the components of the composition. Excipients may be selected from, for example, the group consisting of buffering agents, solubilizing agents, tonicity agents, chelating agents, antioxidants, antimicrobial agents, and preservatives.

A composition for use according the third aspect of the present invention may be administered alone or in combination with other treatments, either simultaneously or sequentially dependent upon the condition to be treated.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### Examples

### Example 1—Lactobacillus crispatus Colonizes Skin of Healthy Individual and is Lost in Atopic Eczema and Psoriasis

To identify microbes which are beneficial to skin function, remodelling and anti-inflammatory potential, skin microbiome datasets were analysed from over 300 individuals including Healthy volunteers, patients with Atopic Eczema, and Psoriasis. Reads from the 16S sequencing were aligned using the QUIME software. Operational Taxonomic Unit (OTU) were then integrated with transcriptomic data and pathway analysis performed. Differences in taxonomic abundance was evaluated by Anova statistical test, or by random forest analysis.

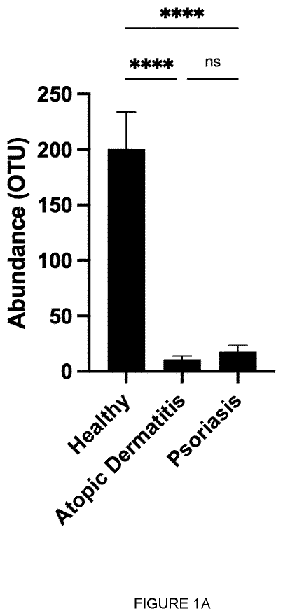

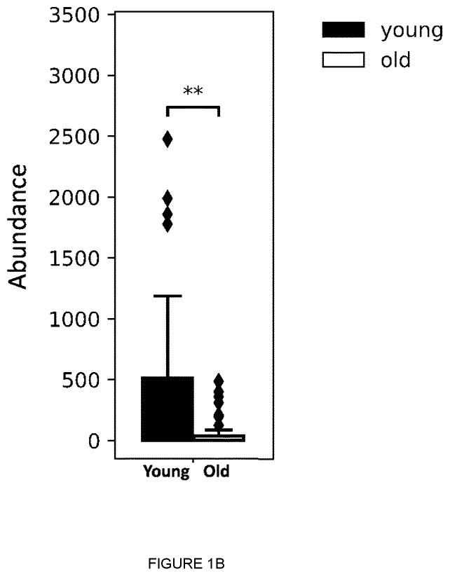

This analysis identified the microbe Lactobacillus crispatus abundantly colonizes the skin of healthy individuals but was massively reduced in patients affected by skin the diseases Atopic Eczema and Psoriasis (FIG. 1A). This data suggests a beneficial function of L. crispatus in the treatment of Atopic Eczema, Psoriasis and associated pathology such as dermatitis, skin infection, dry skin, skin hyperplasia.

### Example 2—L. crispatus Colonizes Skin of Young Individuals and is Reduced in Elderly Individuals

The above data analysis methodology described in Example 1 was applied to identify microbes which have a beneficial anti-aging function by comparing the commensals present on the skin of young individuals (19 to 25 years of age) vs older individuals (44 to 77 years of age). This analysis revealed the abundance of L. crispatus on the skin tends to decrease with age, with the group of older healthy individuals showing significantly decreased L. crispatus colonisation compared to the skin of younger healthy individuals (FIG. 1B). Such a correlation is suggestive of a role for L. crispatus in preventing or delaying skin aging.

### Example 3—L. crispatus is Associated with Anti-Aging and Anti-Inflammatory Pathways

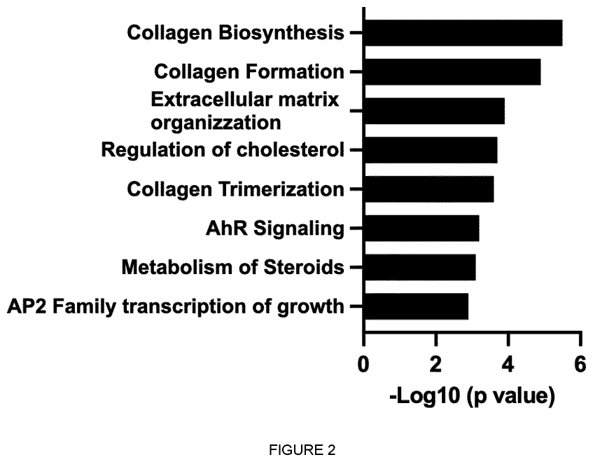

To study how L. crispatus can positively affect skin function, segregation of 25% high vs 25% low or absent L. crispatus colonized healthy donors was performed. Genes associated with these two group were then analysed by means of the online analysis tools Reactome and KEGG pathway. This analysis revealed L. Crispatus is associated with pathways associated with: collagen formation, collagen biosynthesis and retinol, suggesting a role for L. crispatus in prevention of skin aging and remodelling (FIG. 2). It was further revealed that L. crispatus regulates pathways involved in anti-inflammatory responses by regulating gene expression of, for example, the skin protective transcription factor Aryl-hydrocarbon receptor, and the inflammatory pathways AP-1 and AP-2 (FIG. 2).

### Example 4—L. crispatus Lysates Activate the Aryl-Hydrocarbon-Receptor Pathway

The transcription Factor AhR has been shown to mediate anti-inflammatory and skin protective effects when activated in keratinocytes. To evaluate if L. crispatus would activate Aryl-hydrocarbor-Receptor, the activation of the downstream pathway of AhR was evaluated in keratinocytes exposed to L. crispatus lysate.

The cytochrome isoform CYP1A1 and the AhR repressor (AhRR) genes are under the direct control of the transcription factor AhR. Primary keratinocytes were treated with L. crispatus lysates in presence or absence of a AhR inhibitor for 12 hours and the induction of CYP1A1 and AhRR were evaluated by RT-PCR.

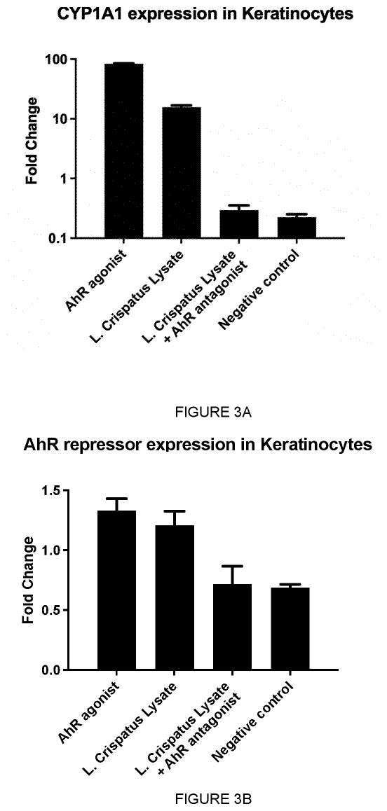

Both CYP1A1 and AhRR were induced by the L. crispatus lysates and the effect was abrogated by the presence of AhR inhibitor (FIG. 3A and FIG. 3B). This data suggest AhR is engaged by L. Crispatus lysates in keratinocytes and mediate anti-inflammatory and skin remodelling effects. L. crispatus lysates were also seen to promote keratinocyte proliferation in these assays (data not shown).

### Example 5—L. crispatus Lysates Protect Keratinocytes from Irritant Induced Cell Death

Next, the functional effects of L. Crispatus in keratinocytes was investigated using an in vitro irritation model using primary adult keratinocytes (NHEK).

Primary keratinocytes from passage 2 to 3 were cultured in 96 well plates at a seed concentration of 7,000 cells/well and cultured in complete keratinocyte medium. Once sub confluent (90 to 95%) cells were pulsed for 2 hours with L. crispatus lysates or active principles. After 2 hours pre incubation, cells were treated with SDS. 2 hours later MTT was added to the culture and incubated for further 2 hours for a total incubation time of 6 hours. After incubation, the MTT assay was developed according to manufacturing instruction and O.D was measured in a 96 well plate reader to determine the survival rate of the keratinocytes.

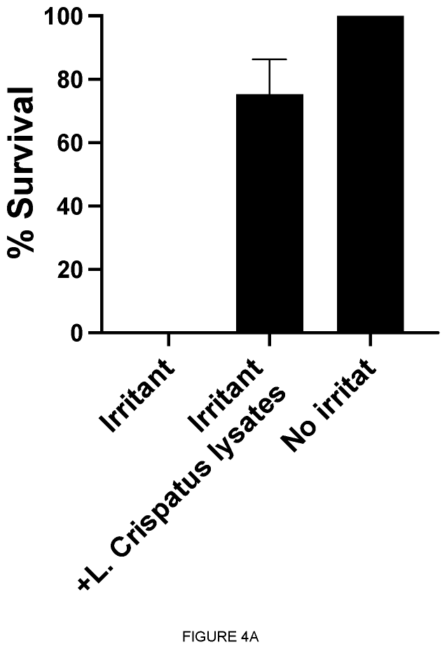

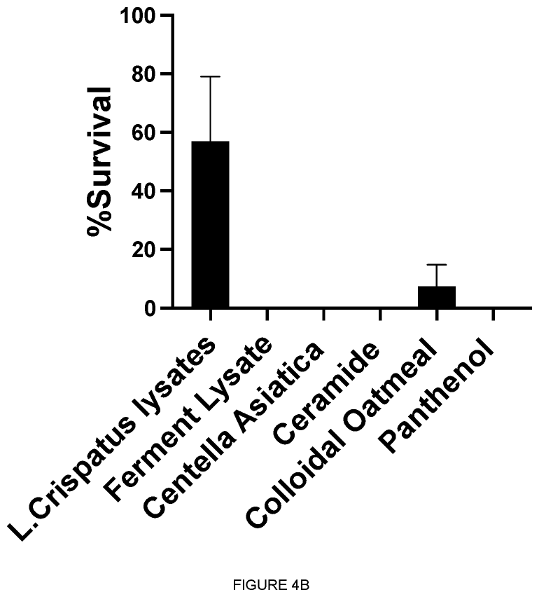

Treatment with SDS alone in the above irritation model was shown to be highly toxic leading to a 0% survival rate following 6 to 8 hours incubation with SDS (FIG. 4A). Pre-treatment with L. crispatus lysates dramatically improved survival rates, with an average survival rate of 70% compared to control cells. These data suggest that the L. Crispatus lysates protect skin cells against irritant mediated cell death.

To compare the potency of L. crispatus lysate protection against commonly used skin care active principles, L. crispatus lysates were compared with commercially available Panthenol, Centella asiatica, Lactobacillus ferment lysates, Colloidal Oatmeal and Ceramides in the in vitro irritant test. L. crispatus but not any other active principle protected the keratinocytes from SDS induced cell damage (FIG. 4B).

These data suggest that L. crispatus lysate have superior potency with respect to keratinocytes protection from cell damage and that its mechanism of action is different form the other active principles or active principles.

### Example 6—Lactobacillus crispatus Lysates Reduces SDS Induced Inflammation in the Skin of Healthy Volunteers and Showed No Irritative Profile

To evaluate if L. crispatus lysates would have a significant skin protective function in vivo, a skin irritant test was performed in healthy volunteers.

Each individual was treated with the following solutions:
Solution 1: SDS 4% containing SDS solubilized in PBS
Solution 2: SDS 4% containing L. Crispatus lysate diluted 1:2 from the original preparation
Solution 3: L. Crispatus lysates diluted 1:2 from the original preparation.

The same amount of each solution was applied to a plaster with a dimension of 1 cm². The plasters containing the solutions were then applied on the forearm of healthy donors in occlusion to increase penetration of the solution into the skin. 24 hours after application, the plasters were removed, pictures were recorded and irritation was evaluated by blinded dermatologists.

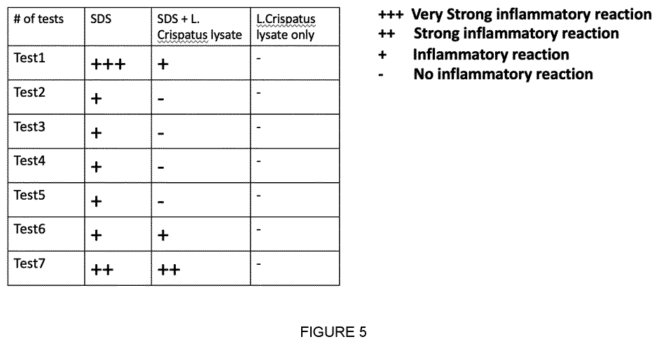

SDS alone induced skin inflammation in all the individuals, with features of erythema and immune cell infiltration in the skin (FIG. 5). L. crispatus lysates strongly reduced the SDS inflammatory response in 5/7 test subjects. Conversely, L. crispatus lysates alone didn't induce any skin irritation in any of the test subjects suggesting the lysates are non-irritant.

Overall, these data suggest strong anti-inflammatory and skin protective effects of L. crispatus lysates in human skin.

### Example 7—Lactobacillus crispatus Lysates Promote Resolution of Skin Inflammation and Reduce Pruritus

To evaluate if L. crispatus lysate would have a significant anti-inflammatory function in skin disorders, volunteers affected by Eczema, Irritative dermatitis, Genital irritation, and genital balanitis were treated with a composition for topical application containing L. crispatus lysates. Every volunteer applied the composition one to three times a day on the affected area of the skin. All the volunteers (100%) reported improvement in the skin condition, redness and associated pruritus within 24 hours of the application. Overall these data suggest an anti-inflammatory and anti-pruritus activity of L. Crispatus lysate in multiple skin disorders.

### References

A number of publications are cited above in order to more fully describe and disclose the invention and the state of the art to which the invention pertains. Full citations for these references are provided below. The entirety of each of these references is incorporated herein.

Agache, A., 2016. Agache's measuring the skin. Cham: Springer

Gottschalck, T. and McEwen, G., 2004. International cosmetic ingredient dictionary and handbook. Washington, D.C.: Cosmetic, Toiletry, and Fragrance Association.

Harry, R. and Rieger, M., 2000. Harry's cosmeticology. New York: Chemical Pub. Co.

Haas, K., Weighardt, H., Deenen, R., Köhrer, K., Clausen, B., Zahner, S., Boukamp, P., Bloch, W., Krutmann, J. and Esser, C., 2016. Aryl Hydrocarbon Receptor in Keratinocytes Is Essential for Murine Skin Barrier Integrity. Journal of Investigative Dermatology, 136(11), pp.2260-2269

Moore & Holdeman, 1970 (Brygoo & Aladame, 1953) Lactobacillus crispatus in GBIF Secretariat (2021). GBIF Backbone Taxonomy. Checklist dataset https://doi.org/10.15468/39omei accessed via GBIF.org on 2021 Nov. 4.

Remington, J. and Gennaro, A., 2000. Remington the science and practice of pharmacy. Baltimore, Md.: Lippincott Williams & Wilkins.

Williams, D. and Schmitt, W., 1996. Chemistry and Technology of the Cosmetics and Toiletries Industry. Dordrecht: Springer Netherlands.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A composition for topical administration comprising;
a lysate of Lactobacillus crispatus or an active fraction thereof; and/or
a population of Lactobacillus crispatus;
wherein the lysate, active fraction and/or population is present in an amount effective for improving the appearance of skin relative to skin prior to administration of the composition, improving skin health, preventing or treating skin inflammatory disorders, preventing or treating skin irritation, preventing or reducing skin pruritus, preventing or treating genital irritation, vaginitis or balanitis, treating or preventing acne, delaying or preventing skin aging, and/or decreasing the apparent age of skin relative to skin prior to administration of the composition.

2. The composition according to claim 1, wherein the composition further comprises one or more physiologically acceptable carriers, excipients, preservatives, fillers, anti-caking agents, anti-foaming agents, bulking agents, thickeners, gellants, emulsifying agents, surfactants, film-forming agents, propellants, pH adjusters, neutralising agents or stabilisers.

3. The composition according to claim 2, wherein the one or more excipients is selected from isopropanol, hydroxypropylcellulose, glyceryl stearate, cetylstearyl alcohol, glycerol, cetyl alcohol liquid petrolatum, isopropyl myristate, Methyl POB, Propyl POB.

4. The composition according to claim 1, wherein the composition further comprises one or more physiologically acceptable active agents selected from an astringent, anti-oxidant, anti-microbial, anti-fungal, chelating agent, topical analgesic, anti-aging agent, anti-wrinkle agent, skin lightening agent, skin conditioning agent, anti-irritant, anti-inflammatory, anti-cellulite agent, emollient, sun protecting agent, self-tanning agent, biologically active peptide.

5. The composition according to claim 1, wherein the composition is in the form of a cream, lotion, gel, mousse, solid stick, spray, ointment, or milk.

6. The composition according to claim 1, wherein the lysate, active fraction, or population is present in an amount sufficient to alter the expression of genes involved in one or more of collagen biosynthesis, collagen formation, extracellular matrix organisation, collagen chain trimerization, Aryl-hydrocarbon signalling, anti-apoptotic molecules, cells proliferation and inflammation when applied to skin cells, keratinocytes, fibroblasts and skin immune cells in vitro or in vivo.

7. The composition according to claim 1, wherein the population, lysate or active fraction thereof is present in amount sufficient to activate the Aryl-hydrocarbon-receptor pathway when applied to skin cells, keratinocytes, fibroblasts and skin immune cells in vitro or in vivo.

8. The composition according to claim 1, wherein the lysate, fraction, or population is present in amount sufficient to promote keratinocyte proliferation in vitro or in vivo, protect keratinocytes from irritant induced cell death in vitro or in vivo, or prevent or reduce irritant-induced inflammation of skin.

9. The composition according to claim 1, wherein the lysate is at a concentration from 0.0001% to 100% by weight, from 0.001% to 75% by weight, 0.01% to 50% by weight, or from 0.1% to 25% by weight in relation to the total weight of the composition.

10. The composition according to claim 1, wherein the population is at a concentration from 0.0001% to 100% by weight, from 0.001% to 75% by weight, 0.01% to 50% by weight, or from 0.1% to 25% by weight in relation to the total weight of the composition.

11. A cosmetic method comprising applying the composition according to claim 1 to the skin of a subject, wherein the method:
i) improves the appearance of skin relative to the appearance of the skin before application of the composition;
ii) improves skin health
iii) prevents or reduces skin irritation,
iv) prevents or reduces pruritus;
v) prevents or reduces genital irritation, including vaginitis and balanitis;
vi) prevents or reduces acne; and/or
vii) delays and/or reverses skin aging.

12. The cosmetic method of claim 11, wherein skin appearance is improved by;
a) increasing the firmness, plumpness, smoothness, skin hydration; and/or
b) decreasing the redness, flakiness, and/or roughness of the skin.

13. A method of:
i) improving skin health;
ii) Preventing or treating skin inflammation
iii) preventing or treating skin irritation,
iv) preventing or treating pruritus;
v) preventing or treating genital irritation, vaginitis, or balanitis;
vi) preventing or treating acne; and/or
vii) delaying or preventing skin aging or reversing the apparent age of skin relative to skin prior to administration of the composition of claim 1;
wherein the method comprises topical administration of a therapeutically effective amount of the composition to the skin of a subject in need thereof.

14. A method of treating a subject with the composition according to claim 1, wherein the composition improves skin health; prevents or treats skin inflammation, skin irritation; prevents or treats pruritus; prevents or treats genital irritation, vaginitis, or balanitis, treats or prevents acne, prevents or delays skin aging, or decreasing the apparent age of skin relative to skin prior to administration of the composition.

15. The method according to claim 14, wherein the subject has an inflammatory skin disease, irritative skin disease or dermatitis.

16. The method according to claim 15, wherein the inflammatory skin disease or dermatitis is irritant contact dermatitis, atopic dermatitis, eczema, atopic eczema, psoriasis, and/or skin irritation caused by an inflammatory skin irritant.

17. The cosmetic method of claim 11, wherein the composition alters the expression of genes involved in one or more of collagen biosynthesis, collagen formation, extracellular matrix organisation, collagen chain trimerization, Aryl-hydrocarbon signalling, anti-apoptotic molecules, cell proliferation and inflammation.

18. The cosmetic method according to claim 17, wherein the composition increases the expression of genes involved in one or more of collagen biosynthesis, collagen formation, extracellular matrix organisation, collagen chain trimerization, Aryl-hydrocarbon signalling, anti-inflammatory or anti-apoptotic molecules, and/or decreases expression of pro-inflammatory molecules.

19. The cosmetic method according to claim 17, wherein the composition activates the Aryl-hydrocarbon-receptor pathway.

20. A method for altering the expression of genes involved in one or more of collagen biosynthesis, collagen formation, extracellular matrix organisation, collagen chain trimerization, Aryl-hydrocarbon signalling, anti-apoptotic molecules, cell proliferation and inflammation in a subject, the method comprising topical administration of the composition according to claim 1.

21. A method for activating the Aryl-hydrocarbon-receptor pathway in a subject, the method comprising topical administration of the composition according to claim 1.

22. The method according to claim 20, wherein the subject is human.
